(19) United States
(12) Patent Application Publication
(10) Pub. No.: US 2022/0323248 A1
(43) Pub. Date: Oct. 13, 2022

(54) INTRANASAL SPLINT COMPRISING ANGLED WING STRUCTURE

(71) Applicant: Ahmet ISLAM, Istanbul (TR)

(72) Inventor: Ahmet ISLAM, Istanbul (TR)

(21) Appl. No.: 17/639,473

(22) PCT Filed: Sep. 5, 2019
(86) PCT No.: PCT/TR2019/050727

§ 371 Date: Mar. 1, 2022

(51) Int. Cl. A61F 5/08

(52) U.S. Cl. CPC A61F 5/08

(57) ABSTRACT

Disclosed is a therapeutic intranasal splint maintaining valve angle by supporting the nasal septum and lateral nasal wall, thus keeping the nasal passage open, and helping to obtain a positive result from performed nose surgery.

### TECHNICAL FIELD

The invention relates to therapeutic intranasal splint maintaining valve angle by supporting the nasal septum and lateral nasal wall (the side wall of the nose), thus keeping the nasal passage open, and helping to obtain good result from performed nose surgery.

In particular, the invention relates to an intranasal splint preventing formation of septal hematoma and adhesion after nose surgeries such as rhinoplasty, septoplasty, nasal valve surgery, repair of nasal septum perforation and opening of synechia.

### BACKGROUND ART

Today, more than half of adults suffer from chronic nasal congestion and mouth breathing while sleeping. Chronic nasal congestion and mouth breathing while sleeping lead to dry throat, impaired quality of sleeping, frequent upper respiratory tract infections and a decline in effort capacity while reducing the quality of life. The most common causes of nasal obstruction are nasal septum deviation, concha (turbinate) hypertrophy, nasal valve collapse, and adhesions within the nose. These conditions may be structural or may also be due to nasal traumas, infections and other inflammatory diseases encountered during any period of life. For permanent treatment, surgery is often required. Besides the functional problems in the nose, there may be aesthetic problems. Nasal aesthetic surgeries, which are commonly called rhinoplasty, are applied to correct aesthetic problems in the nose. After both functional and aesthetic nose surgeries, splints are placed inside the nose.

The purposes of splints are to provide septal support, to prevent adhesions between the nasal septum (the middle part of the nose) and lateral nasal wall and to prevent developing of septal hematoma after surgery. For these purposes, today splints such as Doyle nasal splints, intranasal splints, bivalve nasal splints (silicone nose tampon) which are made of medical silicone are used after nose surgeries. These splints stay inside the nose for 3-4 days on average after surgery.

Although some of the splints used in prior art comprises an integral airway, often within a day following surgery, the airway is obstructed by mucus or clot, thus the patient has to breathe through the mouth.

Some of the splint types used in prior art are flat and all parts have same constant thickness. When these splints are fixed to the anterior part of the nasal septum, posterior ends of the splints do not create enough pressure on the septum. For this reason, they cannot prevent developing hematoma at the posterior part of the septum. In fact, the posterior ends of these splints curl to lateral and obstruct the nasal passage.

Furthermore, in prior art flexible structured winged splints are used only for preventing adhesions. In order to support the lateral nasal wall by the wings in these splints, a tampon that is placed in the nasal passage is needed. Nasal passage is obstructed by the use of these tampons and the patient cannot continue nasal breathing.

In known applications, during the postoperative period, the nasal obstruction continues until the removal of the splint. This decreases the patient comfort and increases the risk of complications, especially in patients with heart-lung diseases and obstructive sleep apnea. In addition, patients who have obstructive sleep apnea and use PAP (Positive Airway Pressure) device cannot use this device with none of the splints present in prior art.

As a result, due to the above-mentioned drawbacks and the inadequacy of the existing solutions, an improvement in the technical field has been required.

### BRIEF DESCRIPTION OF THE INVENTION

The invention is inspired by the existing circumstances and aims to solve the above-mentioned drawbacks.

The main purpose of the invention is providing the patient to breathe through the nose after nose surgeries by keeping the nasal passage open.

Another purpose of the invention is to prevent complications as postoperative bleeding, septal hematoma, and adhesions after the surgery.

Another purpose of the invention is to provide support by creating pressure on each region of the nasal septum that is contacted.

Another purpose of the invention is enabling irrigation with physiological saline solution in the early postoperative period to remove mucus and clots inside the nose, thus achieving rapid mucosal healing.

To fulfill the above-mentioned purposes, the invention is an intranasal splint to be placed into nasal cavity after nose surgeries, maintaining valve angle by supporting nasal septum and lateral nasal wall, thus providing to keep nasal pasage open,
a body to support nasal septum,
   comprising a concave inner surface to be faced to nasal septum and a convex outer surface to be faced to nasal passage, formed between an anterior end, a posterior end, an upper edge and a lower edge;
   having a thickness decreasing while crossing from central part to the anterior end, the posterior end, the upper edge and the lower edge;
a wing
   connected to the upper edge of the body in a region close to the anterior end in a way opening to the lateral by a valve angle; maintaining valve angle between the nasal septum and the lateral wall by supporting the lateral nasal wall, and providing to keep nasal pasage open.

The structural and characteristic features and all advantages of the invention will be understood clearly by the figures and the detailed description made by referring to these figures. Therefore the evaluation should be made by taking these figures and the detailed explanation into consideration.

### BRIEF DESCRIPTION OF THE FIGURES

FIG. 1 is the front view of the invented intranasal splints.

FIG. 2 is the top view of the invented intranasal splints.

FIG. 3 is the bottom view of the invented intranasal splints.

FIG. 4 is the rear view of the invented intranasal splints.

FIG. 5 shows the alternative views of the invented intranasal splints.

FIG. 6a shows the intranasal view of invented intranasal splints before suturing.

FIG. 6b shows the intranasal view of invented intranasal splints after suturing.

### BRIEF DESCRIPTION OF THE REFERENCES

A. Intranasal splint

1. Body
1.1. Anterior end
1.2. Posterior end
1.3. Upper edge
1.4. Lower edge
1.5. Inner surface
1.6. Outer surface
1.7. Central part

2. Wing

α. Valve angle

### DETAILED DESCRIPTION OF THE INVENTION

In this detailed description, the preferred structures of a splint (A) of the invention are described only for a better understanding of the subject.

The invented intranasal splint (A) supports nasal septum and nasal lateral wall by being fixed to the septum by suturing within the nose after nose surgeries. In this way, it provides to keep the nasal passage open by maintaining the valve angle between the nasal septum and the lateral nasal wall. As the nasal passage remains open, the patient's nasal breathing continues after nose surgery and patient comfort is provided.

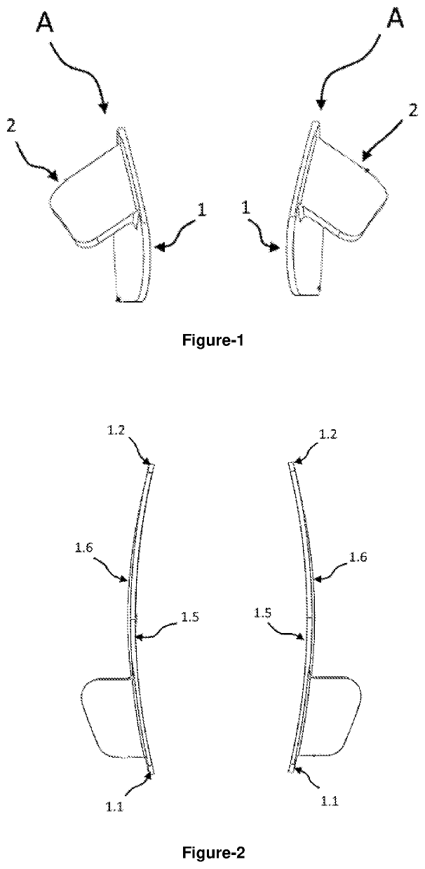

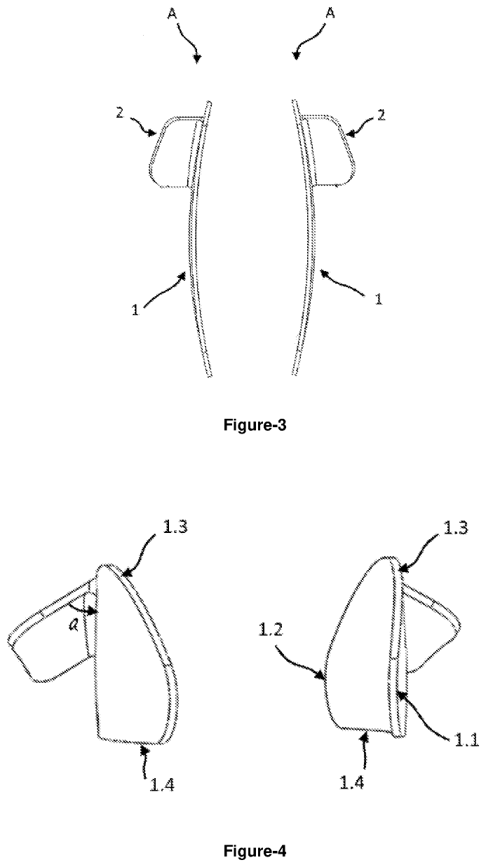

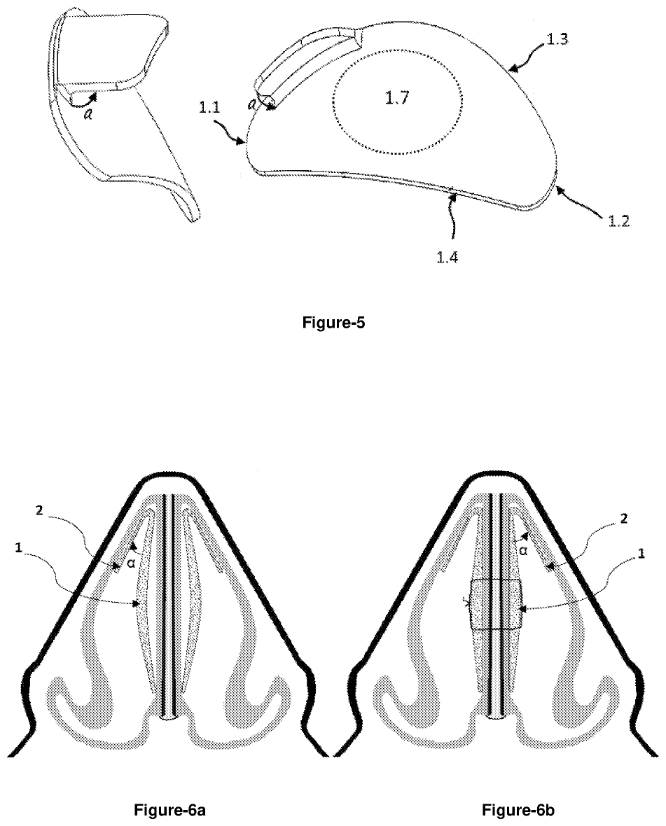

Intranasal splint (A) shown in a representative view in FIG. 1 comprises a body (1) and a wing (2) essentially. The intranasal splint (A) is preferably produced integrally from medical grade silicone by die-casting or three-dimensional printing.

The body (1) comprises concave inner surface (1.5) facing to the nasal septum and a convex outer surface (1.6) facing to the nasal passage, formed between an anterior end (1.1), a posterior end (1.2), an upper edge (1.3) and a lower edge (1.4).

Thichkness of the body (1) tapers while crossing from central part (1.7) to the anterior end (1.1), the posterior end (1.2), the upper edge (1.3) and the lower edge (1.4). The body (1) is made of medical silicone with enough degree of hardness to protect the forms of concave inner surface (1.5) and the convex outer surface (1.6). Thanks to the concave and convex surfaces in horizontal and vertical axises and the central part (1.7) of the body (1) which is thicker than the ends and edges; a surface tension occurs at the posterior end (1.2) towards the septum when the intranasal splint (A) is being fixed with suture to the nasal septum and this tension prevents curling of the posterior end (1.2) towards to the lateral. Thus, each part of the body (1) applies pressure to the nasal septum. Thanks to the pressure occurred development of nasal septal hematoma is prevented.

The length between the anterior end (1.1) and the posterior end (1.2) of the body (1) is greater than the length between the upper edge (1.3) and the lower edge (1.4). In this way, the intranasal splint (A) can support a large portion of the nasal septum. The anterior end (1.1) and the posterior end (1.2) of the body (1) is preferably formed rounded.

The wing (2) is integrally connected (continuous) to the upper edge (1.3) of the body (1) from a region close to the front end (1.1) in a way opening to the lateral by a valve angle (α). The wing (2) supports the lateral wall upwardly, thus maintains the valve angle between the nasal septum and the lateral nasal wall and provides keeping nasal passage open.

The wing (2) is also made of medical silicone with enough degree of hardness to protect the desired valve angle (α).

The valve angle (α) between the wing (2) and the body (1) is preferably between 15 and 90 degrees.

Intranasal splints (A) are used as pairs being placed in the right and left nasal cavity. Right and left intranasal splints (A) are mirror images of each other. The bodies (1) and the wings (2) can be cut to the desired size and applied to the patient's nose in appropriate size. Both intranasal splints (A) are inserted into the nose postoperatively and the bodies (1) are fixed with suture to the nasal septum (FIG. 6a-6b). Fixed bodies (1) provide support by forming pressure onto the nasal septum. Thus, after nose surgeries formation of complications such as bleeding, septal hematoma and adhesion are prevented in postoperative period. When the bodies (1) are being fixed to the septum, thanks to the concave/convex surfaces, the posterior ends (1.2) apply enough pressure on the nasal septum so curling to the lateral does not occur. However, the wings (2) support the lateral nasal wall from below and keeps the valve angle (α) wide so that the nasal passage remains open. Thus, in the postoperative period, the patient can continue breathing through the nose.

1. An intranasal splint to be placed into a nasal cavity after nose surgeries, maintaining a valve angle by supporting the nasal septum and the lateral nasal wall, thus providing to keep the nasal pasage open, the intranasal splint comprising:
a body to support the nasal septum, comprising a concave inner surface to be faced to the nasal septum and a convex outer surface to be faced to the nasal passage, formed between an anterior end, a posterior end, an upper edge and a lower edge; the body having a thickness decreasing while crossing from central part to the anterior end, the posterior end, the upper edge and the lower edge;
a wing connected to the upper edge of the body in a region close to the anterior end in a way opening to the lateral by a valve angle (α); maintaining valve angle between the nasal septum and the lateral wall by supporting the lateral nasal wall, and providing to keep the nasal pasage open.

2. The intranasal splint according to claim 1, wherein the valve angle (α) between the body and the wing is between 15° and 90°.

3. The intranasal splint according to claim 1, wherein the intranasal splint is made of medical grade silicone.

4. The intranasal splint according to claim 1, wherein the length between the anterior end and the posterior end is greater than the length between the upper edge and the lower edge.
